(19) United States
(12) Patent Application Publication
(10) Pub. No.: US 2005/0228227 A1
(43) Pub. Date: Oct. 13, 2005

(54) Multilevel endoscope stem

(75) Inventor: Michael Weber, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/100,010

(22) Filed: Apr. 6, 2005

(30) Foreign Application Priority Data
Apr. 8, 2004 (DE) 102004018128.4

(51) Int. Cl. A61B 1/00

(52) U.S. Cl. 600/128; 600/138

(57) ABSTRACT

An endoscope stem (1) insertable into the human urethra and comprising zones of different heights on its outer surface (3) is characterized in that the raised zones (2) are configured spaced from each other on said stem's outer surface (3).

### BACKGROUND OF THE INVENTION

1. Field of the Invention

The present invention relates to an endoscope of the kind having a stem insertable into a human urethra.

2. Description of Related Art

Endoscope stems inserted into the human urethra enclose observing optics and also instruments allowing surgery in the bladder or on the prostate. Using the endoscope entails motion such as frequent reciprocation and rotation of the endoscope stem. The ensuing friction between the outer endoscope stem surface and the urethral mucous membrane may injure the urethral wall, especially in lengthy surgery.

As a result it has been known already for decades to apply lubricants to endoscope stems in the urethra in order to lower the friction between the endoscope stem outer surface and the urethra and therefore the danger of injury.

This lubricant is deposited before the endoscope stem is inserted into the urethra, but, when the surgery is lengthy, it will be rubbed off by the motions of the endoscope shaft and thereafter this shaft shall be moved while devoid of lubricant and incurring the attendant noxious effects.

It is known from the German patent document DE 29 12 852 A1 to fit the endoscope stem with a smooth coating of longer life that reduces friction.

The German patent document DE 30 45 237 C2 discloses an endoscope stem fitted with recesses at its outer surface and coated with lubricant. The said recesses serve a supply stores and are meant to substantially delay scraping off the lubricant, but this goal is substantially elusive.

Lastly the German patent document DE 101 11 354 A1 discloses endoscope stems of which the lubricant at the stem surface is constantly replenished. However such designs are unusually complex and demand problematic diametral enlargement.

### SUMMARY OF THE INVENTION

The objective of the present invention is to create a simple endoscope stem which allows use over a substantial time interval while exhibiting good slipperiness.

In the present invention, the endoscope stem comprises raised and separated zones on its outer surface. The stem comes into contact with the urethral wall foremost only by said raised zones. Experiment has shown that the urethral friction is reduced so much that no lubricant is needed at all. Lubricants no longer being required, friction shall be constant even over substantial time intervals of surgery.

### BRIEF DESCRIPTION OF THE DRAWINGS

FIG. 1 of the single, appended drawing shows in illustrative and schematic manner a partial section of a segment of the endoscope stem of the present invention.

### DETAILED DESCRIPTION OF THE INVENTION

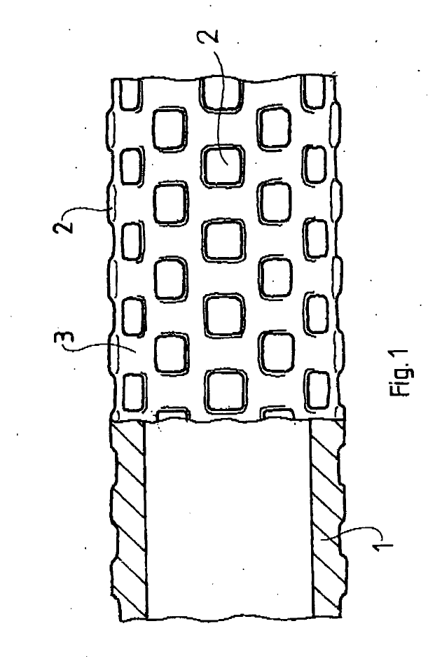

The endoscope stem shown in FIG. 1 illustratively is conventionally made of metal and comprises raised zones 2 on its outer stem surface 3 which are spaced from each other for instance in the regular manner shown. An omitted, urethral wall enclosing the endoscope stem 1 rests at much reduced friction essentially only on the raised zones 2. Compared to an endoscope stem having an uninterrupted smooth surface, the friction encountered in the embodiment of the invention is reduced so much that advantageous friction in the absence of lubricant shall be attained even over a substantial time interval.

In a particular embodiment, the shown endoscope stem for instance may have an outside diameter of about 10 mm. The raised zones 2 may be about ¼ mm2 in area and may project above the adjacent outer surface 3 by a few tenths of a mm. In the preferred and shown embodiment, the gaps between the raised zones 2 correspond to the heights of the raised zones 2. These raised zones advantageously merge as shown in rounded manner into the outer surface 3 to prevent injuring the mucous membrane.

1. An endoscope stem (1) insertable into a human urethra, comprising zones of different heights at its outer surface (3),
wherein the zones (2) are raised zones (2) configured spaced from each other on the outer surface (3).

2. The endoscope stem as claimed in claim 1, wherein the raised zones (2) are uniformly distributed over the outer surface (3).

3. The endoscope stem as claimed in claim 1, wherein the raised zones (2) merge in rounded manner into the outer surface (3).

4. The endoscope stem as claimed in claim 1, wherein the endoscope stem comprises a diameter of approximately about 10 mm, and wherein
the raised zones (2) cover an area of about ¼ mm² and exhibit a height of a few 1/10 mm.
